(19) United States
(12) Patent Application Publication
(10) Pub. No.: US 2015/0291672 A1
(43) Pub. Date: Oct. 15, 2015

(54) Contact Lens Surface Modification with Hyaluronic Acid (HA) Binding Peptide for HA Accumulation and Retention

(71) Applicant: THE JOHNS HOPKINS UNIVERSITY, Baltimore, MD (US)

(72) Inventors: Vincent Beachley, Baltimore, MD (US); Jennifer H. Elisseeff, Baltimore, MD (US); Peter Li, Baltimore, MD (US); Peter John McDonnell, Owings Mills, MD (US); Anirudha Singh, Baltimore, MD (US)

(21) Appl. No.: 14/439,270

(22) PCT Filed: Nov. 1, 2013
(86) PCT No.: PCT/US2013/067970

§ 371 Date: Apr. 29, 2015

Related U.S. Application Data
(60) Provisional application No. 61/721,196, filed on Nov. 1, 2012.

(51) Int. Cl. C07K 14/435; A61K 9/00

(52) U.S. Cl. CPC C07K 14/435; A61K 9/0051

(57) ABSTRACT

An embodiment in accordance with the present invention provides a device and method for providing HA to the ocular environment. A contact lens according to the present invention is treated at its surface with a HA binding peptide. The HA binding peptide can be covalently bonded to a functional group on the surface of the contact lens, such as OH, COOH, or NH2. The lens can then be pretreated with HA for immediate increased wearer comfort upon insertion of the lens. As HA is washed away or degraded from the surface of the lens, the HA binding peptide remains and therefore HA can be replenished from endogenous or exogenous sources.

### CROSS-REFERENCE TO RELATED APPLICATION

This application claims the benefit of U.S. Provisional Patent Application No. 61/721,196, filed Nov. 1, 2012, which is incorporated by reference herein, in its entirety.

### FIELD OF THE INVENTION

The present invention relates generally to ophthalmology. More particularly the present invention relates to a device and method for providing moisture to the eye.

### BACKGROUND OF THE INVENTION

Contact lens irritation reduces the comfort of lens wear and decreases the length of time the lenses can be worn. Many attempts have been made in order to increase a wearer's comfort. One such attempt includes delivering hyaluronic acid (HA) to lubricate the wearer's eye via a lubricating eye drop or contact lens solution. HA is a naturally occurring polysaccharide that has excellent water retention properties and can act as a natural lubricant. Unfortunately, the concentration of HA at the surface of the contact lens, when applied using an eye drop or lens solution, may not be sufficiently high to produce maximum benefit. Additionally, soluble HA, such as that in an eye drop or contact lens solution, is rapidly cleared from the eye at a rate of approximately 99% in one hour. Another method of getting HA into the ocular environment is to incorporate it directly into the lens. However, HA directly incorporated into a lens is not self-renewable and may be degraded quickly in vivo.

It would therefore be advantageous to provide a device and method for providing moisture to the eye that improve retention and is renewable.

### SUMMARY OF THE INVENTION

The foregoing needs are met, to a great extent, by the present invention, wherein in one aspect a device includes a contact lens. A hyaluronic acid binding peptide is coupled to the contact lens.

In accordance with another aspect of the present invention, the contact lens further includes a first surface configured for contact with an eyeball, a second surface configured for contact with an eyelid, and a bulk disposed between the first surface and the second surface. The hyaluronic acid biding peptide can be coupled to the first surface of the contact lens, the second surface of the contact lens, and/or to the bulk of the contact lens. The hyaluronic acid binding peptide can be covalently bonded to the contact lens. The hyaluronic acid binding peptide can also be configured to bind endogenous and supplemental sources of hyaluronic acid. Additionally, the hyaluronic acid binding peptide can bind a new hyaluronic acid molecule after a first hyaluronic acid molecule is cleared. The contact lens is formed from a hydrogel, and can include an exposed functional group selected from one of a group consisting of OH, COOH, and NH₂.

In accordance with another aspect of the present invention, a method for moisturizing an eye includes applying a hyaluronic acid binding peptide to a surface. The method also includes providing a source of hyaluronic acid for binding to the hyaluronic acid binding peptide. The surface can be one of a surface of the eye or a surface of a contact lens.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIG. 1A illustrates a view of a contact lens treated with a hyaluronic acid binding peptide according to an embodiment of the present invention.

FIG. 1B illustrates a schematic diagram of a contact lens as it is treated with HABpep and HA, according to an embodiment of the present invention.

FIG. 1C illustrates a schematic diagram of a contact lens as it is treated with PEG and HA, according to an embodiment of the present invention.

FIG. 2 illustrates FITC labeled HABpep visualized on the contact lens surface for EDC coupled and control groups.

FIG. 3A illustrates fluorescence images of unmodified lenses (i and ii) and modified lenses (iii, iv, v).

FIG. 3B illustrates a graphical view of fluorescent intensity of contact lenses conjugated with FITC-HABpep with various concentrations (i-vi).

FIG. 4A illustrates images of fluorescence-based visualization of a PEGylated contact lens via an amine functional group in views i-iv. FIG. 4B illustrates a graphical of fluorescent intensity of contact lenses conjugated with Fluorescein-PEG with various concentrations over i-iv (0 mg/mL to 6 mg/mL).

FIG. 5A illustrates images of contact lenses with no spacer (from left to right: control-HA, control+HA, HABpep+HA (0.05/1.0 mg/mL), and HABpep+HA (0.5/1.0 mg/mL)).

FIG. 5B illustrates images of contact lenses with a spacer (from left to right: control-HA, control+HA, HABpep+HA (0.05/1.0 mg/mL), and HABpep+HA (0.5/1.0 mg/mL)).

FIG. 6A illustrates a schematic diagram of an experiment setup for showing that water retention is enhanced by bound HA via HABpep in contact lenses.

FIG. 6B illustrates a graphical view of evaporation rate for lenses having different treatments.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

An embodiment in accordance with the present invention provides a device and method for providing HA to the ocular environment. A contact lens according to the present invention is treated at its surface with a HA binding peptide. The HA binding peptide can be covalently bonded to a functional group on the surface of the contact lens, such as OH, COOH, or NH₂. The lens can then be pretreated with HA for immediate increased wearer comfort upon insertion of the lens. As HA is washed away or degraded from the surface of the lens, the HA binding peptide remains and therefore HA can be replenished from both endogenous or exogenous sources.

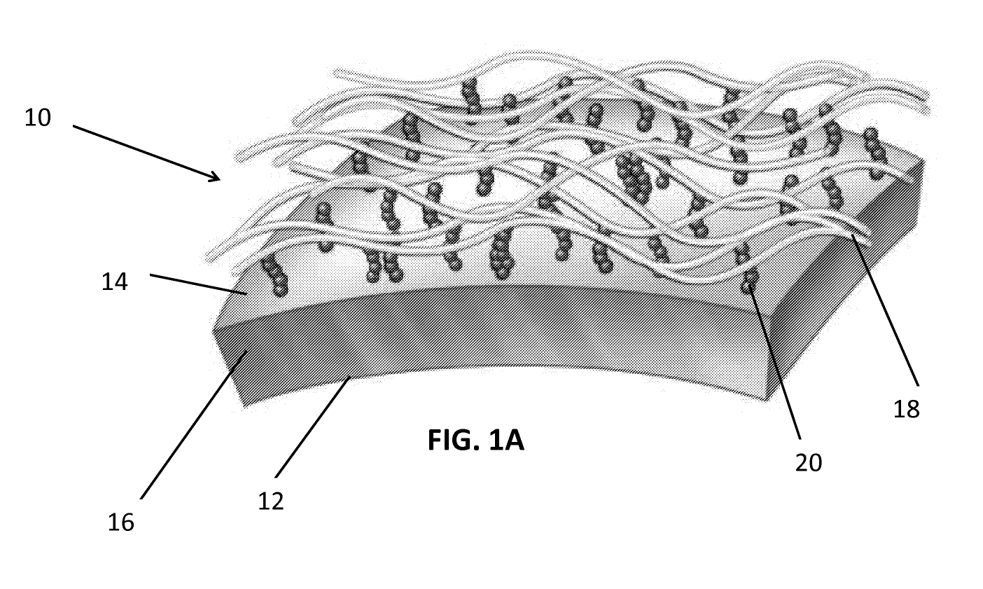

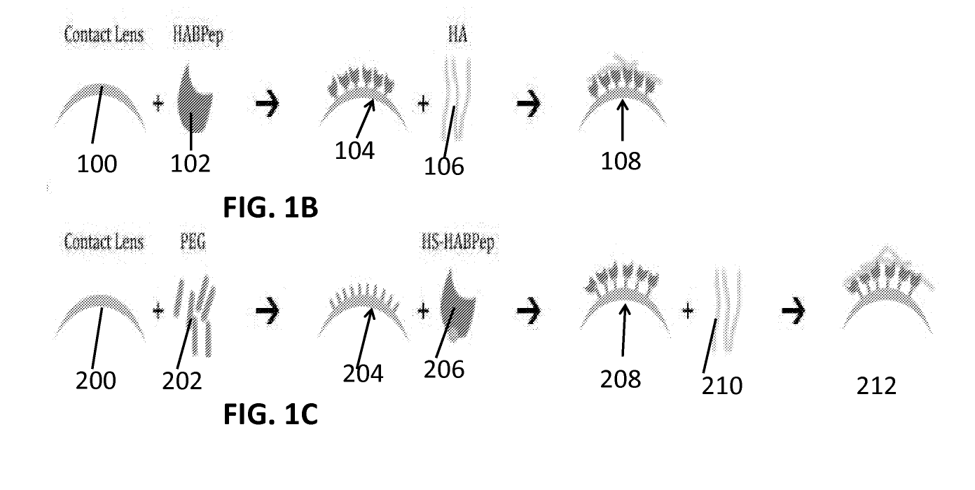

FIG. 1A illustrates a view of a contact lens treated with a hyaluronic acid binding peptide according to an embodiment of the present invention. The contact lens 10 includes a first surface 12 configured to come into contact with the eyeball of the wearer. A second surface 14 of the contact lens 10 is disposed opposite the first surface 12 and is configured to contact an inner surface of the eyelid of the wearer. A bulk 16 of the lens 10 is disposed between the first surface 12 and the second surface 14. The first surface 12 and the second surface 14 can be coated with a HA binding peptide. Either the first surface 12 or the second surface 14 can be coated independently or the two surfaces can both be coated. The lens 10 is preferably formed from a hydrogel having an exposed functional group such as an OH, COOH, or NH₂. The HA binding peptide can then be covalently bonded directly to the lens 10. Alternately, the HA binding peptide can be incorporated into the bulk 16 of the lens 10, independently or in conjunction with binding the HA binding peptide to the first surface 12 and the second surface 14. The HA binding peptide can bind either endogenous or exogenous HA in the ocular environment. The contact lens 10 can come pre-treated with HA 18, and additional HA can be added to the ocular environment or to the contact lens using eye-drops, contact solution, or any other means of lens treatment known to or conceivable by one of skill in the art. Therefore, as HA is washed away from the contact surface the binding peptide remains attached and the surface can be replenished as new HA attaches to the peptide. In some embodiments, as illustrated in FIG. 1A, the lens 10 can also be treated with a PEG spacer 20.

FIG. 1B illustrates a schematic diagram of a contact lens as it is treated with HABpep and HA, according to an embodiment of the present invention. FIG. 1B illustrates the contact lens 100 being combined with the HABpep 102 to yield a contact lens coated with HABpep 104. The contact lens coated with HABpep 104 is combined with HA 106 to yield a contact lens coated with HABpep and bound to HA 108. FIG. 1C illustrates a schematic diagram of a contact lens as it is treated with PEG and HA, according to an embodiment of the present invention. FIG. 1C illustrates the contact lens 200 being combined with the PEG 202 to yield a contact lens coated with PEG 204. The contact lens coated with HABpep 204 is combined with HS-HABpep 206 to yield a contact lens coated with PEG and HS-HABpep 208. The contact lens coated with PEG and HS-HABpep 208 is combined with HA 210 to yield a contact lens coated with PEG, HS-HABpep, and HA 212.

More particularly with respect to the HA binding peptide 18 discussed above with respect to FIGS. 1A-1C, a peptide sequence was previously discovered by phage display, which non-covalently binds hyaluronic acid (HA). The peptide sequence for this binding peptide referred to as, “Pep-1,” is GAHWQFNALTVR. In the present invention, “Pep-1” is used as the HA binding peptide (HABpep) and is covalently linked to the surface of commercial contact lenses. The HABpep coating will capture and retain HA at the contact surface at higher levels than seen without an HApep coating. Furthermore, increased levels of HA at the lens surface will result in improvements in water retention and lubrication leading to improved comfort for contact lens wearers.

It is also notable that while, “Pep-1 and HABpep are discussed above, the invention is not limited to this formulation of HA binding peptide. This invention, which is to covalently bind HABpep to contact lenses can be attained by a wide variety of chemical modifications. First, different functional groups can be added to the terminal end of the peptide during synthesis to facilitate subsequent covalent binding to the available free functional groups contact surface. In addition, the contact surface can be modified with specific functional groups to facilitate covalent binding to HABpep. Several schemes to covalently bond HABpep to the surface of commercial contact lenses (PureVision, Baush & Lomb) have been investigated. HABpep has been synthesized with thiol and amine groups at the terminal ends. L-Photo-Leucine has been reacted on the surface of lenses to add free amine and carboxyl groups. Covalent binding reactions have been performed using 1-ethyl-3-(3-dimethylaminopropyl) carbodiimide (EDC) and 1,1′-carbonyldiimidazole (CDI) chemistry to show that HABPep can be adhered to the contact lens surface. It should further be noted that any means for binding the HABpep to the surface of the contact lens known to or conceivable by one of skill in the art can be used.

The following examples are included merely as an illustration of the present method and are not intended to be considered limiting. These examples are one of many possible applications of the methods described above. Any other suitable application of the above described methods known to or conceivable by one of skill in the art could also be created and used.

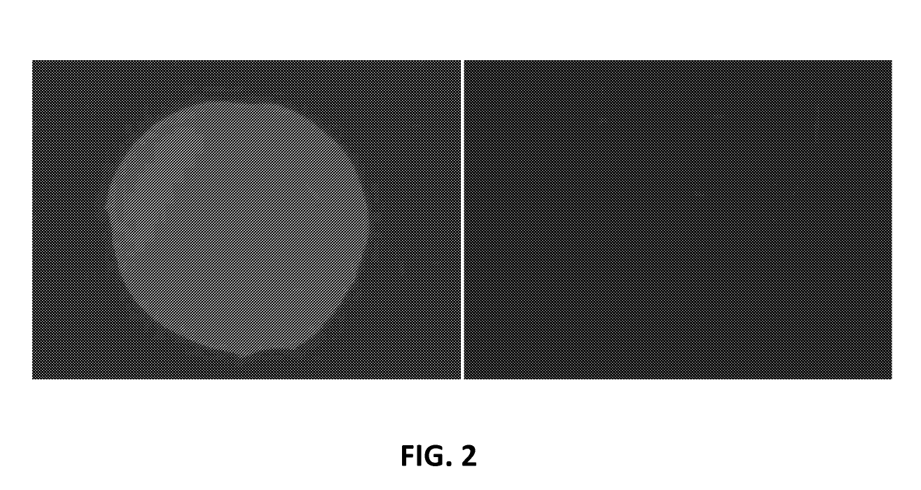

In one example, which is not to be considered limiting, but merely an illustration of one way to bind HABpep to the surface of the contact lens, EDC binding was performed by dissolving EDC and N-Hydroxysuccinimide (NHS) in 0.05M MES solution (pH 5.6) at 2 mg/ml and 1.2 mg/ml respectively. Contact lenses are then incubated in the solution at 37° C. for ten minutes and then moved to a second solution that contains HABpep at 1 mg/ml in addition. Lenses are incubated with HABpep for 4 hrs at 37° C. The lenses are washed thoroughly in PBS to remove any unbound HABpep. The selective covalent attachment of HABpep to the contact surface via the EDC binding reaction was confirmed using a fluorescently labeled HABpep sequence. FIG. 2 shows FITC labeled HABpep visualized on the contact lens surface for EDC coupled and control groups. More particularly, FIG. 2 illustrates FITC labeled HABpep is visualized on the contact surface after EDC coupling on the left. A control lens on the right soaked in FITC labeled HABpep without EDC coupling is compared on the right. Both lenses were thoroughly washed after HABpep treatment.

Preparation of HA-Binding Coatings on Contact Lens Surfaces without a Spacer:

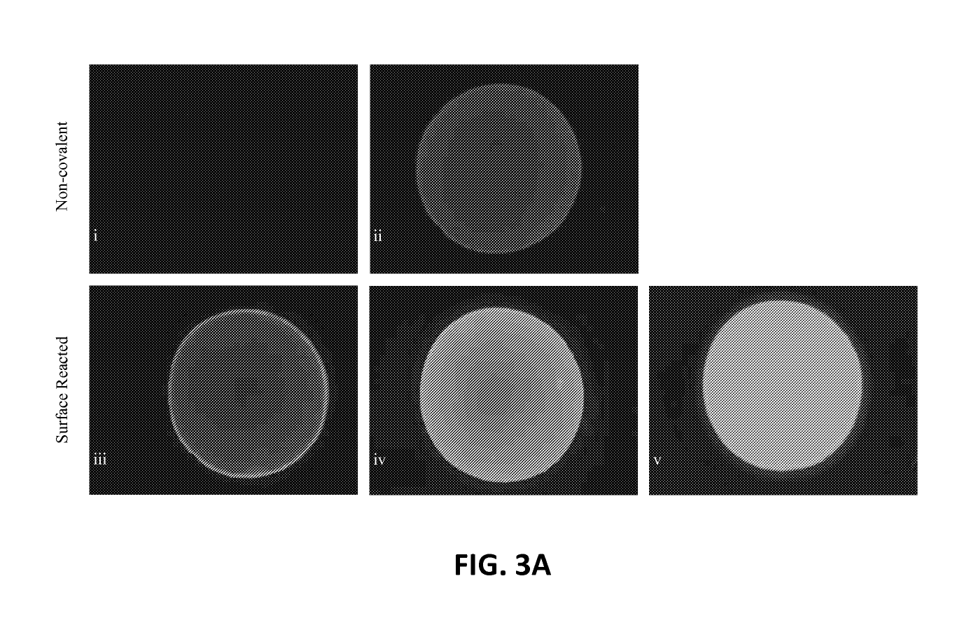

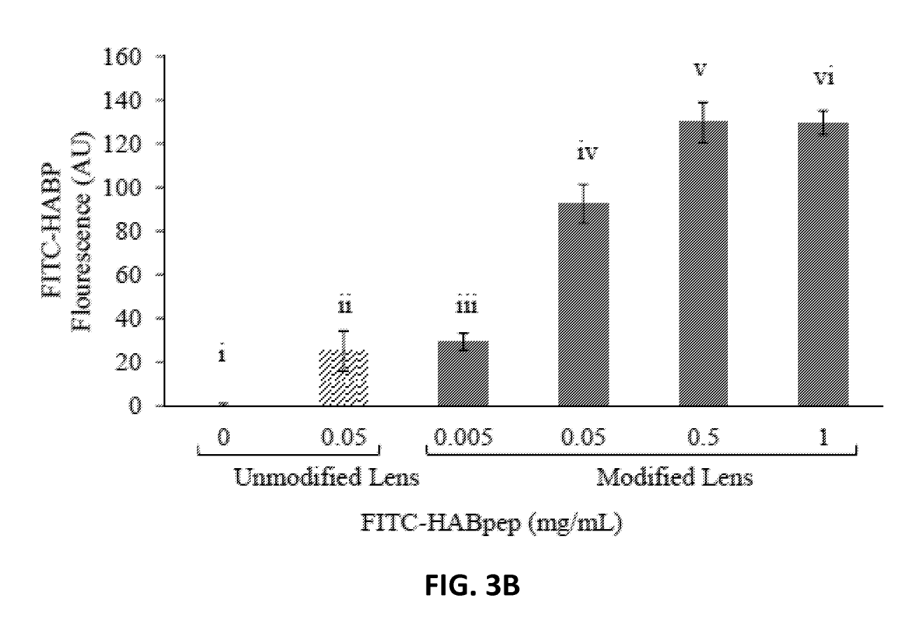

Contact lenses (PureVision®, balafilcon A, 36% water from Bausch and Lomb, N.Y.) were cut using 3.0 mm or 4.5 mm biopsy punches, which were added to MES buffer solutions (pH 5.4) containing N-(3-Dimethylaminopropyl)-N′-ethylcarbodiimide hydrochloride or EDC (Sigma-Aldrich, St. Louis; 3.0 mg/mL) and N-hydroxysuccinimide or NHS (Sigma-Aldrich, St. Louis; 2.4 mg/mL). After 10 min of activation, the samples were transferred to PBS (pH 7.4; Life Technologies) solutions of either HABPep (GAHWQFNALTVR, ChinaPeptides, Shanghai) or FITC-HABPep (ChinaPeptides, Shanghai) of varying concentrations (0, 0.005, 0.05, 0.5, 1.0, and 1.5 mg/mL) and stirred for 24 h at room temperature. The cut samples were vigorously washed with PBS to removed unreacted HABpep or FITC-HABpep. FIGS. 3A and 3B illustrate fluorescence-based visualization of an HABpep modified contact lens via direct conjugation and no spacer. FIG. 3A illustrates fluorescence images of unmodified lenses (i and ii) and modified lenses (iii, iv, v). FIG. 3B illustrates a graphical view of fluorescent intensity of contact lenses conjugated with FITC-HABpep with various concentrations (i-vi). The lenses with great concentration of FITC-HABpep show relatively instense fluorescence compared to the contact lens with no FITC-HABpep.

Preparation of HA-Binding Coatings on Contact Lens Surfaces with a Spacer.

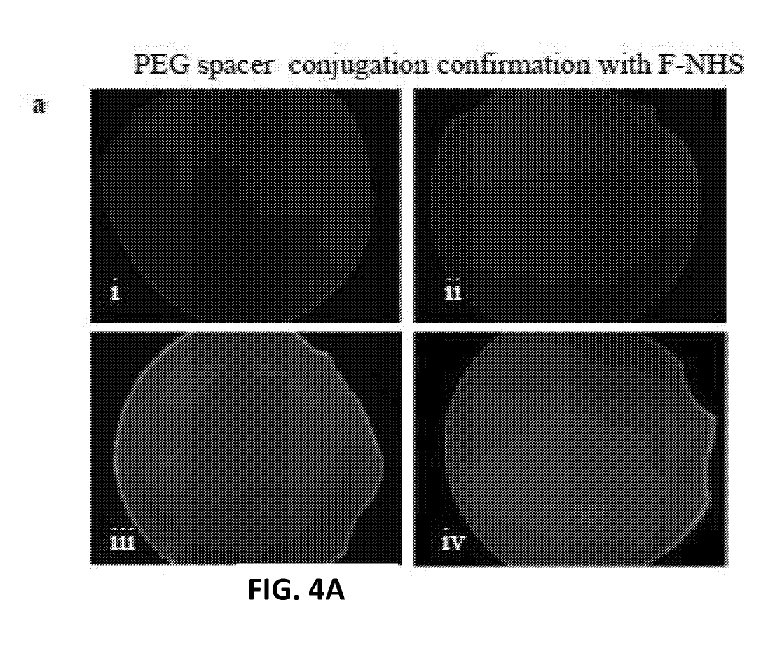

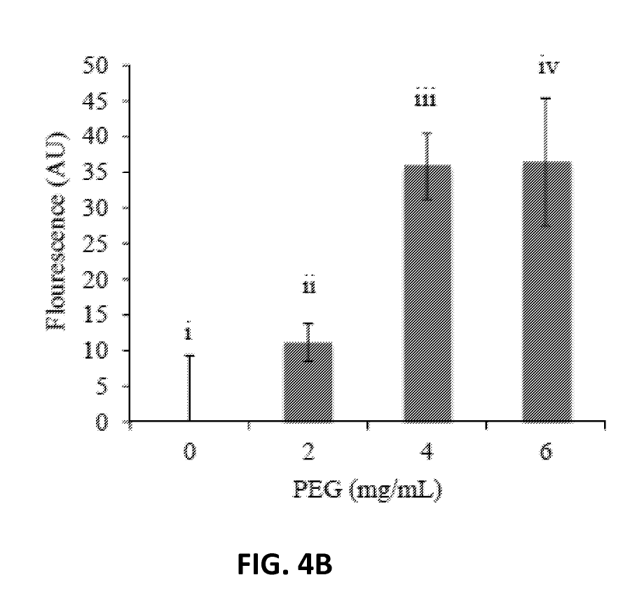

HABpep was conjugated to the contact lenses through a heterobifunctional poly(ethylene glycol) (PEG) spacer. First, contact lens samples were modified with amine functional groups by stirring them in MES buffer solutions (pH 5.4) containing EDC (3.0 mg/mL) and NHS (2.4 mg/mL) followed by transferring them into a PBS buffer solution (pH 7.4) containing an excess of ethylene diamine (10 mg/mL). After 4 h of reaction, contact lens samples were vigorously washed with PBS (pH 7.4). A heterofunctional PEG spacer, MAL-PEG-NHS (3.5 kDa, JenKem) was dissolved to 5 mM in 50 mM sodium bicarbonate, pH 7.5, and added to the contact lens samples. The NHS groups were allowed to react with the amines on the contact lens surface for 1 h. Following thorough washes in buffer to remove unreacted crosslinker, a 1.5 mM solution of C-HABpep (CRRDDGAHWQFNALTVR) was added to the surface to react with maleimide groups for an additional 1 h. Samples were washed vigorously to remove unreacted peptide, yielding HABpep modified contact lenses. FIG. 4A illustrates images of fluorescence-based visualization of a PEGylated contact lens via an amine functional group in views i-iv. FIG. 4B illustrates a graphical of fluorescent intensity of contact lenses conjugated with Fluorescein-PEG with various concentrations over i-iv (0 mg/mL to 6 mg/mL). The lenses with greater concentration of Fluorescein-PEG showed greater intensity than the control with no Fluorescein-PEG.

Fluorescence Visualization and Measurements of the HA-Bound Contact Lens Surface.

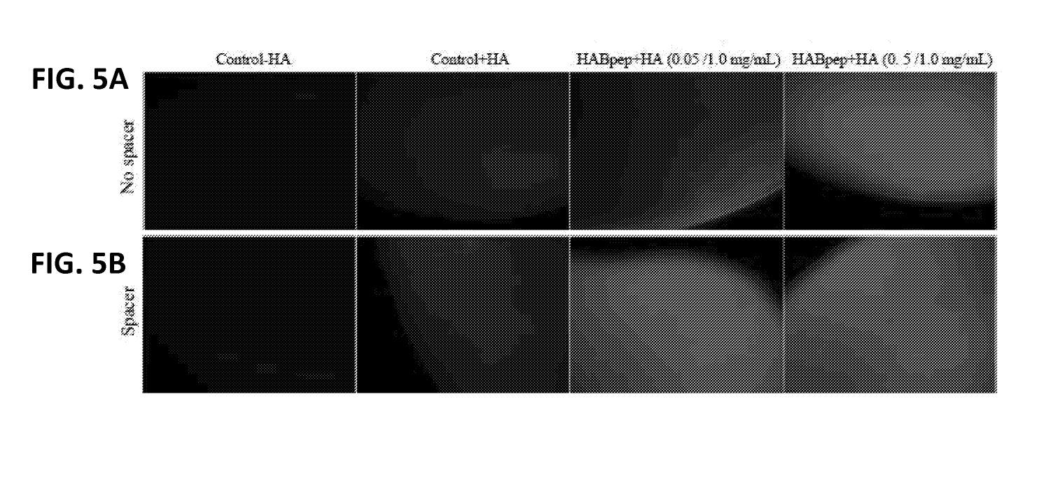

HABpep-modified contact lenses were added to a solution of HA-rhodamine (CreativePEGWorks; 1.0 mg/mL) and kept on a shaker for 24 h. After vigorous washing with PBS three times for 24 h, fluorescence images were taken by Zeiss Discovery V2 imaging microscope and processed with ImageJ. To measure HA absorption or binding on both unmodified and modified contact lenses, the contact lens samples were submerged into 200 μL of fluorescently labeled HA in a 96-well round bottom plate and the fluorescence was measured by a plate reader. A standard curve was created using known HA concentrations. The following day, 150 μL of the HA soak solution from each well is relocated and the fluorescence was measured. HA concentration is calculated from 150 μL of the standard assay. To measure FITC-HABpep binding, the contact lenses were imaged on a Zeiss microscope before the overnight HABpep treatment. The lenses were rinsed vigorously and then imaged again. The brightness of a representative box was calculated with ImageJ both before and after the treatment. Results were normalized to the control. To measure the HABpep saturation, contact lenses were treated at 0, 0.5, 1.0, 1.5 mg/mL FITC-HABpep. To quantify HA binding versus HABpep, contact lenses were treated with 0, 0.005, 0.05, 0.5, and 1.0 mg/mL HABpep and soaked in 1 mg/mL HA. To measure HA retention, the contact lenses from HA quantification were soaked in 200 μL HBSS and left on a shaker. The solution was changed each day and the fluorescence of the 200 μL wash buffer was measured. HA concentration was calculated from a standard assay. FIG. 5A illustrates images of contact lenses with no spacer (from left to right: control-HA, control+HA, HABpep+HA (0.05/1.0 mg/mL), and HABpep+HA (0.5/1.0 mg/mL)). FIG. 5B illustrates images of contact lenses with a spacer (from left to right: control-HA, control+HA, HABpep+HA (0.05/1.0 mg/mL), and HABpep+HA (0.5/1.0 mg/mL)).

Water Retention Studies:

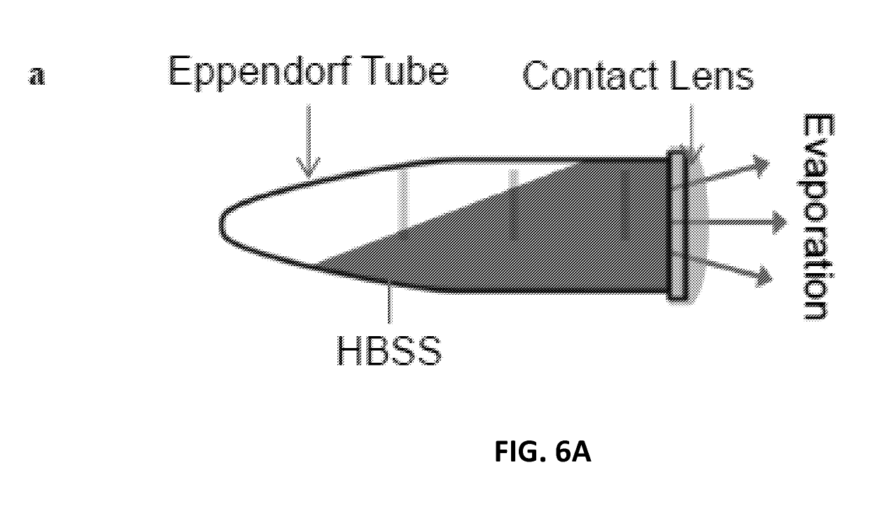

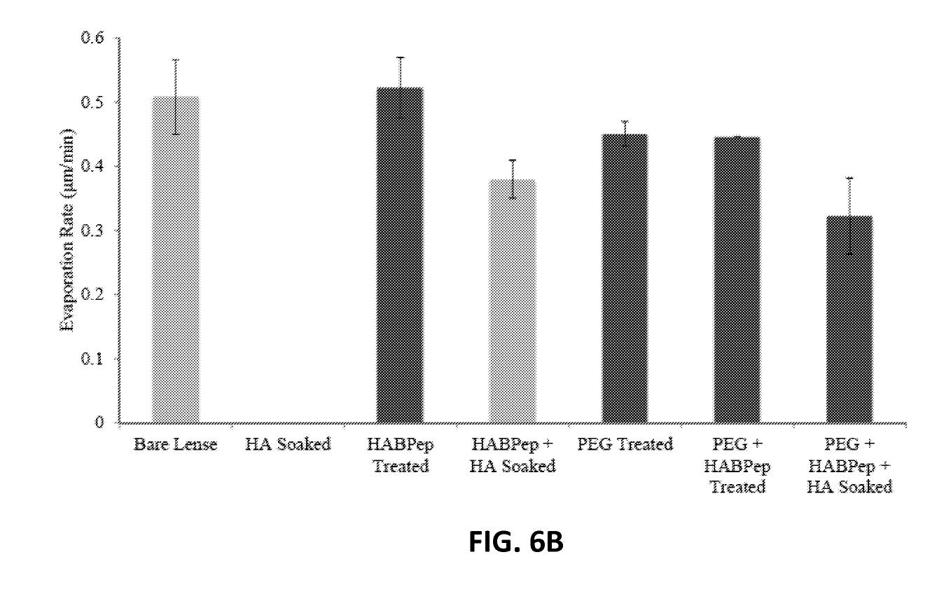

To measure the rate of evaporation of water from the contact lens, an evaporation cell was designed by cutting the cap and hinge off of a 1.5 mL SealRite® microcentrifuge tube (USA Scientific, Ocala, Fla.) with the inside and outside diameters of 1.0 cm and 1.3 cm, respectively (as illustrated in FIG. 6A). The cell was filled up with 1.2 mL of Hanks' Balanced Salt solution or HBSS (Invitrogen, CA) and the contact lens was glued to the rim of the cell with instant Krazy glue (Elmer's Products, OH). The cell was tested for leaks and the solution was gravimetrically moved to completely cover the lens. The cell was gently placed on its side, keeping the inside contact surface completely hydrated, into an analytical balance. The weight of the cell was recorded to 5 decimal places at the start and every 5 min for 50 min. FIG. 6A illustrates a schematic diagram of an experiment setup for showing that water retention is enhanced by bound HA via HABpep in contact lenses. FIG. 6B illustrates a graphical view of evaporation rate for lenses having different treatments. As shown in FIG. 6B the lenses treated with HA had the lower evaporation rates.

It should be noted that although the invention is described with respect to contact lenses it is possible that the HA binding peptide can be bound to a different form of delivery device known to or conceivable by one of skill in the art. Alternately, the HA binding peptide could also be bound directly to the eye. As technology related to contact lenses also changes, it is conceivable that the device and method be modified to accommodate lenses made from different materials and new manufacturing techniques. Versatility in both peptide modification and chemical attachment methods will allow optimization of binding techniques for specific lens materials and other manufacturing requirements. New techniques in manufacturing could also allow the HA binding peptide to be incorporated directly into the surface or the bulk of the lens. All of these possibilities are considered within the scope of the present invention.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

1. A device comprising:
a contact lens; and
a hyaluronic acid binding peptide coupled to the contact lens.

2. The device of claim 1 wherein the contact lens further comprises a first surface configured for contact with an eyeball, a second surface configured for contact with an eyelid, and a bulk disposed between the first surface and the second surface.

3. The device of claim 2 further comprising the hyaluronic acid biding peptide being coupled to the first surface of the contact lens.

4. The device of claim 2 further comprising the hyaluronic acid binding peptide being coupled to the second surface of the contact lens.

5. The device of claim 2 further comprising the hyaluronic acid binding peptide being coupled to the bulk of the contact lens.

6. The device of claim 2 further comprising the hyaluronic acid binding peptide being coupled to the first and second surfaces of the contact lens.

7. The device of claim 1 further comprising the hyaluronic acid binding peptide being covalently bonded to the contact lens.

8. The device of claim 1 further comprising the hyaluronic acid binding peptide being configured to bind one of endogenous and supplemental sources of hyaluronic acid.

9. The device of claim 1 further comprising the hyaluronic acid binding peptide can bind a new hyaluronic acid molecule after a first hyaluronic acid molecule is cleared.

10. The device of claim 1 wherein the contact lens is formed from a hydrogel.

11. The device of claim 1 wherein the contact lens further comprises an exposed functional group selected from one of a group consisting of OH, COOH, and NH₂.

12. A method for moisturizing an eye comprising:
applying a hyaluronic acid binding peptide to a surface; and
providing a source of hyaluronic acid for binding to the hyaluronic acid binding peptide.

13. The method of claim 12 further comprising applying the hyaluronic acid binding peptide to a surface of the eye.

14. The method of claim 12 further comprising applying the hyaluronic acid binding peptide to a surface of a contact lens.

15. The method of claim 14 further comprising applying the hyaluronic acid binding peptide to the surface of the contact lens using direct conjugation.

16. The method of claim 12 further comprising modifying a surface of a contact lens using HABpep.

17. The method of claim 16 further comprising modifying the surface of the contact lens using HABpep and a PEG spacer.

18. The method of claim 12 further comprising modifying a surface of a contact lens with NHS functional groups.

19. The method of claim 12 further comprising modifying a surface of a contact lens with an amine functional group.

20. The method of claim 19 further comprising reacting the surface of the contact lens with a heterofunctional PEG spacer MAL-PEG-NHS to create a thiol reactive PEGylated HA binding site.

21. The method of claim 20 further comprising reacting a thiolated HABpep to a maleimide functionality.

22. The method of claim 21 further comprising exposing the surface of the contact lens to hyaluronic acid.
